(19) United States
(12) Patent Application Publication
(10) Pub. No.: US 2014/0296598 A1
(43) Pub. Date: Oct. 2, 2014

(54) Purification of Aromatic Hydrocarbon Streams

(71) Applicant: ExxonMobil Chemical Patents Inc., Baytown, TX (US)

(72) Inventors: Glenn A. Heeter, The Woodlands, TX (US); John D. Ou, Houston, TX (US); Robert G. Tinger, Friendswood, TX (US)

(21) Appl. No.: 14/186,482

(22) Filed: Feb. 21, 2014

Related U.S. Application Data
(60) Provisional application No. 61/807,067, filed on Apr. 1, 2013.

(51) Int. Cl. C07C 7/12

(52) U.S. Cl. CPC C07C 7/12
USPC 585/446; 585/831; 585/470; 585/477

(57) ABSTRACT

The invention is directed to a process for the removal of olefin impurities from a feedstream comprising greater than equilibrium amounts of paraxylene by contact of the feedstream with a bed of solid acid catalyst to produce a product comprising reduced olefin impurities (when compared with said feedstream), said process comprising at least one of (i) reduced bed temperature on startup, and (ii) reduced flow rate on startup, wherein, in embodiments, there is a reduction in side reactions such as isomerization and/or transalkylation and/or disproportionation of paraxylene, when compared with conventional startup procedures.

### PRIORITY CLAIM

This application claims priority to and the benefit of U.S. Provisional Application No. 61/807,067, filed Apr. 1, 2013, the disclosure of which is incorporated herein by reference in its entirety.

### FIELD OF THE INVENTION

The invention relates to purification of aromatic hydrocarbon streams and more particularly the removal of olefinic compounds from aromatic hydrocarbon streams, especially suitable for removing olefinic side products made in a process for producing paraxylene by alkylation of benzene and/or toluene.

### BACKGROUND OF THE INVENTION

Aromatic streams, which may comprise one or more of benzene, toluene and xylenes (BTX), are used as feedstocks in various petrochemical processes. By way of example, paraxylene obtained from such streams are useful in the production of polyester fibers and films. It is well-known that such streams, derived from processes such as naphtha reforming and thermal cracking (pyrolysis), generally contain undesirable hydrocarbon contaminants including mono-olefins, dienes, styrenes and heavy aromatic compounds such as anthracenes, and that these contaminants must be removed before subsequent processing of the aromatic streams. Less well-known is that in the production of paraxylene by contact of toluene and/or benzene with an alkylating agent such as methanol and/or dimethylether, olefinic impurities such as styrene are produced in side reactions.

Olefinic compounds can be removed from aromatic hydrocarbon streams using solid acids such as clay, aluminosilicates, and zeolites (used interchangeably herein with the term “molecular sieves”). Without wishing to be bound by theory, these materials operate to remove olefin impurities at least in part by alkylating aromatic compounds in the hydrocarbon stream with the olefin, to form heavy aromatics (C9+ aromatic hydrocarbons) that can be removed easily, for instance, by fractionation. See, for instance, U.S. Pat. Nos. 6,368,496; 7,517,824; 7,731,839; 7,744,750; 8,048,295; 8,057,664; 8,216,450; 8,227,654; 8,329,971; and 8,344,200.

Various undesirable side reactions can occur when the feedstream comprising aromatic hydrocarbons and olefinic impurities contacts the solid acid catalysts, including transalkylation, disproportionation, and isomerization of the aromatic hydrocarbons. When the feedstock being treated is a feedstock in a process comprising the making, isolation, or use of paraxylene, the loss of paraxylene (not to mention co-production of benzene and other non-paraxylene aromatics) by any of the aforementioned reactions is a costly problem. In these types of processes, e.g., the production of a paraxylene-rich stream by alkylation of benzene and/or toluene with methanol and/or dimethyl ether, or recovery of paraxylene by a simulated moving bed adsorption apparatus, or the production of purified terephthalic acid from paraxylene, there is a great need for improving the selectivity of zeolite catalysts. It is particularly critical to avoid isomerization of paraxylene in paraxylene-rich feedstreams, i.e., streams wherein paraxylene is present in amounts greater than equilibrium, e.g., greater than 23 wt %, relative to total xylenes.

An improved start up procedure was disclosed in U.S. Pat. No. 8,344,200, wherein the zeolite catalyst is first dewatered and then fresh feedstock is flowed through the reactor at temperatures significantly below normal operating conditions, such as approximately 100° C. or less, for a predetermined period of time, such as between 0.5 to 5 days. Then the temperature of the feedstock is raised to the operating temperature. However, an aromatic hydrocarbon feedstock comprising greater than equilibrium amounts of paraxylene was not discussed and hence the value of low loss of paraxylene was not recognized.

Additionally, relevant recent disclosures include U.S. Pat. No. 8,216,450, wherein reduction of bromine index is achieved by removal of trace olefins and dienes from aromatic feedstocks using start-up conditions outside the ordinary range currently used, such as, in embodiments, the feed is heated and contacts the zeolite catalyst above temperatures currently used, such as about 210° C., and the temperature is gradually increased to between about 240 and 300° C. at the end of the cycle.

The present inventors have surprisingly discovered that side reactions in the removal of olefins including styrene from an aromatic hydrocarbon feedstream comprising greater than equilibrium amounts of paraxylene can be reduced or eliminated by employing a startup in which the bed temperature is reduced and/or the flow rate is increased.

### SUMMARY OF THE INVENTION

The invention is directed to a process for the removal of olefin impurities from an aromatic hydrocarbon feedstream comprising greater than equilibrium amounts of paraxylene and olefin impurities by contact of the feedstream with a bed of solid acid catalyst to produce a product comprising reduced olefin impurities, said process comprising at least one of (i) reduced bed temperature on startup, and (ii) increased flow rate on startup, when compared with normal operating conditions, wherein, in embodiments, there is a reduction in side reactions such as isomerization and/or transalkylation and/or disproportionation of paraxylene, when compared with conventional startup procedures.

In embodiments, there is a start-up procedure wherein a catalyst comprising fresh zeolite, regenerated zeolite, or a combination thereof, in a fixed bed reactor is contacted with a feedstream comprising xylenes and olefin impurities, wherein the conditions of contact include at least one of (i) reduced bed temperature, relative to a predetermined operating bed temperature, and (ii) increased flow rate, relative to a predetermined operating flow rate, for a predetermined period of time, and then the conditions of contact including said predetermined operating bed temperature and predetermined operating flow rate are commenced.

In embodiments, by the use of the method of the present invention, paraxylene selectivity loss is prevented on the first day of start-up, such as within 24 hours or less, or in other embodiments within 48 hours or less, as compared with prior art methods which can take up to 6 months to observe paraxylene selectivity loss.

It is an object of the invention to decrease loss of paraxylene in a process for impurity removal in a paraxylene-enriched feedstream, while at the same time increasing the amount of impurities, particularly styrene, that are removed.

It is another object of the invention to provide a start-up procedure applicable to any para-selective technology such as selective toluene disproportionation, para-selective isomerization, para-selective transalkylation, and the like.

It is yet another object of the invention is to provide a start-up procedure that can be ramped up to normal operating conditions in a short time, such as 24-48 hours.

These and other objects, features, and advantages will become apparent as reference is made to the following detailed description, preferred embodiments, examples, and appended claims.

### BRIEF DESCRIPTION OF THE DRAWINGS

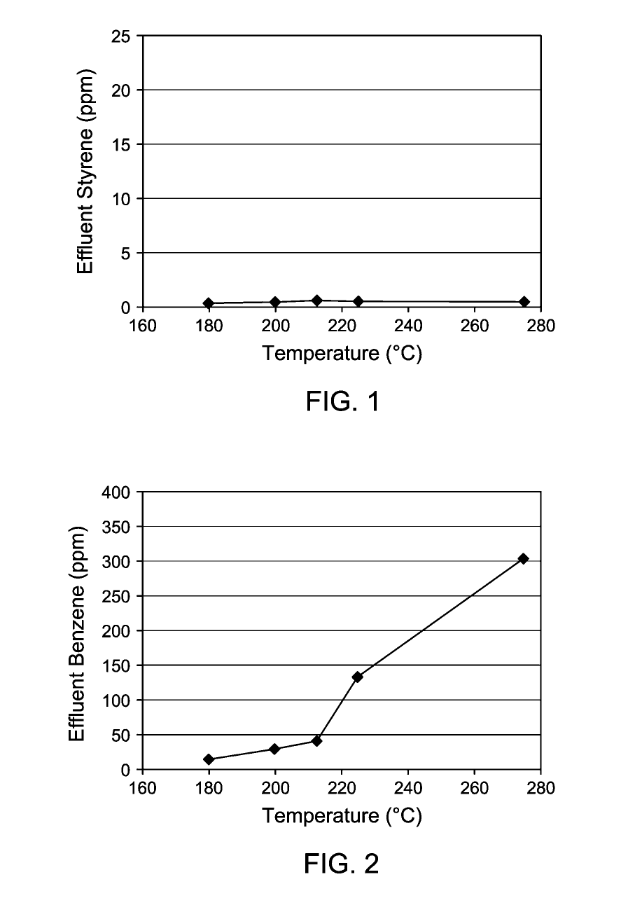

In the accompanying drawings, FIGS. 1-4 show experimental data on impurity removal from paraxylene-enriched feedstreams.

### DETAILED DESCRIPTION

According to the invention, a feedstream comprising paraxylene and at least one olefin impurity, preferably wherein paraxylene is present in said feedstream in an amount greater than 23 wt %, based on total xylene, still more preferably greater than 70 wt %, such as 78 wt %, or 80 wt %, or 86 wt %, or 90 wt %, or 99 wt %, relative to total xylenes, preferably wherein said at least one olefin impurity includes styrene in the amount of at least 10 ppm, such as 50 ppm, or 100 ppm, or 1000 ppm, contacts a catalyst comprising at least one zeolite, preferably a zeolite selected from the MWW framework topology zeolites (IUPAC Commission of Zeolite Nomenclature) as described in “Atlas of Zeolite Framework Types”, eds. W. H. Meier, D. H. Olson and Ch. Baerlocher, Elsevier, Fifth Edition, 2001, such as the MCM-22 family of zeolites, in the presence or absence of hydrogen gas dissolved in said feedstream, to provide a product having a reduced amount of said at least one olefin impurity, preferably wherein said at least one olefin impurity is reduced in an amount of at least 50.0%, more preferably 90.0%, still more preferably 99.0%, and in embodiments wherein said at least one olefin impurity is styrene, reduced in amount to below 5 ppm, preferably below 1 ppm, more preferably below 0.1 ppm and/or undetectable levels by gas chromatographic techniques, and, in embodiments, wherein the loss of paraxylene by said contact is less than 5%, more preferably less than 1%, still more preferably less than 0.1%, still more preferably wherein the loss is not measureable by gas chromatographic techniques, and, in other or additional embodiments wherein the co-production of benzene by said contact is less than 100 ppm, preferably less than 50 ppm, more preferably less than 10 ppm. Ppm is parts per million, relative to total parts of the stream in question.

The zeolite is preferably selected from one or more zeolites having the MWW framework topology (“MWW family”) such as the MCM-22 family. Particularly preferred zeolites include MCM-22, MCM-36, MCM-49, MCM-56, EMM-10, and mixture thereof.

The term “MCM-22 family” (or “material of the MCM-22 family” or “molecular sieve of the MCM-22 family”), as used herein, includes one or more of:
(i) molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, supra);
(ii) molecular sieves made from a common second degree building block, being a 2-dimensional tiling of such MWW framework topology unit cells, forming a monolayer of one unit cell thickness, preferably one c-unit cell thickness;
(iii) molecular sieves made from common second degree building blocks, being layers of one or more than one unit cell thickness, wherein the layer of more than one unit cell thickness is made from stacking, packing, or binding at least two monolayers of one unit cell thickness. The stacking of such second degree building blocks can be in a regular fashion, an irregular fashion, a random fashion, or any combination thereof; and
(iv) molecular sieves made by any regular or random 2-dimensional or 3-dimensional combination of unit cells having the MWW framework topology.

The MCM-22 family materials are characterized by having an X-ray diffraction pattern including d-spacing maxima at 12.4±0.25, 3.57±0.07 and 3.42±0.07 Angstroms (either calcined or as-synthesized). The MCM-22 family materials may also be characterized by having an X-ray diffraction pattern including d-spacing maxima at 12.4+0.25, 6.9+0.15, 3.57±0.07 and 3.42±0.07 Angstroms (either calcined or as-synthesized). The X-ray diffraction data used to characterize the molecular sieve are obtained by standard techniques using the K-alpha doublet of copper as the incident radiation and a diffractometer equipped with a scintillation counter and associated computer as the collection system. Materials belong to the MCM-22 family include MCM-22 (described in U.S. Pat. No. 4,954,325 and U.S. Pat. No. 7,883,686); PSH-3 (described in U.S. Pat. No. 4,439,409); SSZ-25 (described in U.S. Pat. No. 4,826,667); ERB-1 (described in European Patent No. 0293032); ITQ-1 (described in U.S. Pat. No. 6,077,498); ITQ-2 (described in International Patent Publication No. WO97/17290); ITQ-30 (described in International Patent Publication No. WO2005118476); MCM-36 (described in U.S. Pat. No. 5,250,277); MCM-49 (described in U.S. Pat. No. 5,236,575); UZM-8 (described in U.S. Pat. No. 6,756,030); MCM-56 (described in U.S. Pat. No. 5,362,697); EMM-10-P (described in U.S. Pat. No. 7,959,899); and EMM-10 (described in U.S. Pat. Nos. 8,110,176 and 7,842,277).

The zeolite may optionally be co-loaded with one or more other solid acid catalysts such as clay and/or aluminosilicates. The one or more zeolite may optionally be mixed with the one or more clay and/or aluminosilicates or utilized in series or parallel, in separate reactors or the same reactor.

The apparatus is preferably a fixed bed such as of the type well-known in the art, and may be in series and/or parallel with other apparatus also containing the zeolite, alone or co-loaded with another solid acid catalyst such as clay and/or aluminosilicates.

The feedstream acted on by the catalyst may be any aromatic hydrocarbon feedstream containing an olefin impurity, wherein the term “impurity” means an undesired species that is advantageously removed before the feedstream is further acted upon, such as in an adsorption process, such as of the type well-known, e.g., a Parex™ or Eluxyl™ unit, or a crystallization process, or a combination thereof. The present invention is particularly advantageous when employed downstream of a process for the production of a paraxylene-rich stream by the reaction of an alkylating agent such as methanol and/or dimethylether, with toluene and/or benzene, disclosed in, by way of example, U.S. patent application Ser. Nos. 13/483,836 and/or 13/557,605, and/or U.S. Pat. No. 8,399,727, and references cited therein. The present invention is also advantageous when employed downstream of a process comprising para-selective technology, for example selective toluene disproportionation, para-selective isomerization, para-selective transalkylation, and the like, all well-known per se.

The present invention may be better understood by the following detailed examples, which are intended to be representative and not limiting thereof.

The removal of styrene by contact with a solid acid catalyst is carried out in a fixed bed reactor using a paraxylene-enriched feedstream described below. The catalyst comprised 65% MCM-22/35% alumina binder.

FIGS. 1, 2, and 3 show results from a laboratory experiment using said catalyst in a conventional fixed bed apparatus, and contacting a feedstream running at 2.5 WHSV and 265 psig using a xylenes feed with a greater than equilibrium (nominally 79 wt %) concentration of paraxylene that was spiked with 650 ppm styrene. During the initial few days of operation, the reactor temperature was varied over a range of 180-275° C.

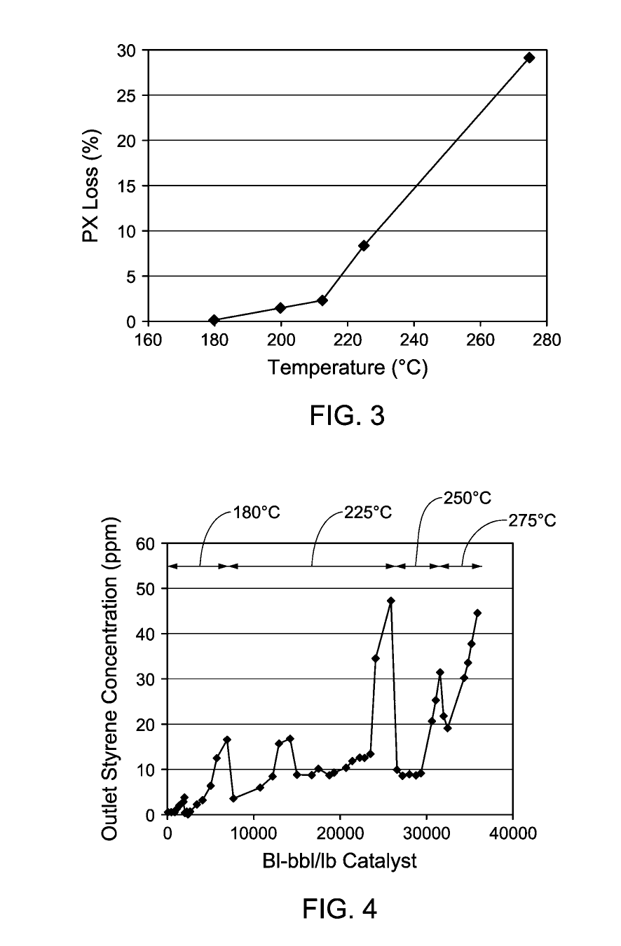

The effect of temperature on the effluent styrene concentration (FIG. 1), the effluent benzene concentration (FIG. 2), and paraxylene (PX) loss due to isomerization (FIG. 3). Measurements were obtained using conventional gas chromatographic methods on the effluent stream and compared with the feedstream concentrations. The effluent styrene concentration was maintained at a consistent <1 ppm level over the entire temperature range, while the effluent benzene concentration and the paraxylene (PX) loss increased substantially as the temperature was increased. This shows that controlling the bed temperature can reduce the undesirable side reactions while maintaining good removal efficiency.

Table 1 shows results at two different WHSVs (A and B in Table 1, below) from a laboratory experiment using a 65% MCM-22/35% alumina binder catalyst running at a bed temperature of 225° C. and 265 psig using a xylenes feed with a greater than equilibrium (nominally 79 wt %) concentration of paraxylene that was spiked with 650 ppm styrene. The reactor was at 9.8 WHSV and the flow rate was reduced to a WHSV of 2.5. The effluent styrene concentration dropped dramatically with the reduction in flow rate but the effluent benzene concentration increased only modestly and the paraxylene (PX) loss was unchanged. This shows that controlling the bed flow rate can maintain good removal efficiency without increasing the undesirable side reactions.

TABLE 1

| | A | B |
|---|---|---|
| WHSV | 9.8 | 2.5 |
| Effluent Styrene Concentration, wppm | 50.7 | 4.1 |
| Effluent Benzene Concentration, wppm | 0.0 | 7.4 |
| Para-xylene loss, % | 0.5 | 0.5 |

FIG. 4 shows results from another laboratory experiment using a 65% MCM-22/35% alumina binder catalyst running at 9.8 WHSV and 265 psig using a xylenes feed with a greater than equilibrium (nominally 79%) concentration of paraxylene that was spiked with 650 ppm styrene. The effluent styrene concentration is plotted in FIG. 4 as a function of the cumulative BI-bbl converted/lb catalyst. (BI is Bromine Index, a well-known measure of olefin impurities; “BI-bbl” is a conventional term used to compare relative catalyst performance to remove bromine reactive species per pound of catalyst. BI-bbl is the BI times the volume of liquid treated, in Barrels (42 U.S. gallons; 1 U.S. bbl oil=158.99 liters)). The bed temperature across the reactor bed is near-isothermal and can be measured, for instance, at the inlet. In this case the feedstream temperature and the bed temperature are the same. Heating of the bed and/or feedstream can be accomplished by conventional heat exchange equipment, e.g., using steam, hot oil, a process stream, or combinations thereof. In this laboratory experiment was initially at 180° C. and was increased whenever the effluent styrene approached or exceeded the target value of 20 ppm. These results show that the styrene conversion can be maintained within a predetermined specification for a time by raising the reactor bed temperature to offset the loss of activity as the catalyst ages.

The invention has been described above with reference to numerous embodiments and specific examples. Many variations will suggest themselves to those skilled in this art in light of the above detailed description. All such obvious variations are within the full intended scope of the appended claims.

Trade names used herein are indicated by a ™ symbol or ® symbol, indicating that the names may be protected by certain trademark rights, e.g., they may be registered trademarks in various jurisdictions. All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted. When numerical lower limits and numerical upper limits are listed herein, ranges from any lower limit to any upper limit are contemplated. While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains.

What is claimed is:

1. A process for the removal of olefin impurities from an aromatic hydrocarbon feedstream comprising paraxylene in greater than equilibrium amounts and olefin impurities, by contact of said feedstream with a solid acid catalyst to produce a product stream comprising reduced olefin impurities, the improvement comprising at least one of the following conditions: (i) reduced bed temperature on startup relative to a predetermined bed temperature at normal operating conditions, and (ii) increased flow rate on startup relative to a predetermined flow rate at normal operating conditions.

2. The process of claim 1, wherein said at least one of conditions (i) and (ii) are continued for a predetermined period of time, and then at least one of said conditions are changed to said normal operating conditions.

3. The process of claim 1, wherein said solid acid catalyst comprises at least one zeolite having the MWW framework topology.

4. The process of claim 1, wherein said olefin impurity includes styrene.

5. The process of claim 1, including a step of alkylation of benzene and/or toluene with methanol and/or dimethylether to produce a first aromatic hydrocarbon feedstream comprising paraxylene and styrene, then contacting said first aromatic feedstream, with or without an intervening step, with said solid acid catalyst, to produce a second aromatic hydrocarbon feedstream having a reduced amount of styrene compared to said first aromatic hydrocarbon feedstream.

6. The process of claim 1, including a step of producing paraxylene selectively to produce a first aromatic feedstream, said step selected from (i) toluene disproportionation, (ii) para-selective isomerization, (iii) para-selective transalkylation, and (iv) mixtures thereof, then contacting said first aromatic feedstream, with or without an intervening step, with said solid acid catalyst, to produce a second aromatic hydrocarbon feedstream.

7. The process according to claim 2, wherein said predetermined period of time is 24 hours or less.

8. The process according to claim 2, wherein said predetermined period of time is 48 hours or less.

9. The process of claim 1, further including, after establishment of normal operating conditions, then raising the reactor temperature to offset loss of activity as the catalyst ages.

10. The process of claim 1, wherein said catalyst is fresh catalyst.

11. The process of claim 1, wherein said catalyst is regenerated catalyst.

12. The process of claim 3, wherein said at least one zeolite includes at least one is zeolite selected from the MCM-22 family.

13. The process of claim 12, wherein said at least one zeolite comprises one selected from the group consisting of MCM-22, MCM-36, MCM-49, MCM-56, EMM-10, and mixtures thereof.
